(19) United States
(12) Patent Application Publication
(10) Pub. No.: US 2004/0162315 A1
(43) Pub. Date: Aug. 19, 2004

(54) Use of compounds for treating conditions resulting from injury to the corneal nerve after LASIK and other ocular surgeries or trauma

(76) Inventors: Mark R. Hellberg, Arlington, TX (US); Iok-Hou Pang, Grand Prairie, TX (US); John M. Yanni, Burleson, TX (US)

Correspondence Address: Teresa J. Schultz, Alcon Research, Ltd., 6201 South Freeway, Q-148, Fort Worth, TX, 76124-2099, US

(21) Appl. No.: 10/775,704

(22) Filed: Feb. 10, 2004

(30) Foreign Application Priority Data
Jul. 23, 2002 (WO) PCT/US02/23871

(51) Int. Cl. A61K 31/445; A61K 31/4184

(52) U.S. Cl. 514/315; 514/394

(57) ABSTRACT

The present invention provides methods for the treatment of conditions resulting from injury to the corneal nerve after LASIK and other ocular surgeries or trauma.

### BACKGROUND OF THE INVENTION

1. Field of the Invention

The present invention is directed to the use of compounds that promote neuron regeneration or neurite outgrowth for the treatment of conditions resulting from injury to corneal nerves following Laser In Situ Keratomileusis (LASIK) or other surgeries where the corneal nerves are damaged.

2. Description of the Related Art

Patients frequently experience a decrease in corneal sensitivity and mild to moderate dry eye after LASIK surgery. In most patients, this is an acute problem lasting for only a few days. However, in a significant number of patients, the problem may persist for several months or more (Yu 2000). This iatrogenic change most likely results from the severing of corneal nerves during surgery (Wilson 2001; Ambrosio & Wilson 2001). Current treatment methods for surgery-induced dry eye include symptomatic reliefs such as the frequent local application of artificial tears, such as Tears Naturale or Bion Tears®, or other artificial moisturizing agents. These treatments reduce discomfort but do not treat the underlying pathology. No acceptable therapy of the decrease in corneal sensitivity is known to the inventors at this time.

Neurotrophic factors are peptide molecules which stimulate or otherwise maintain growth of neuronal tissue. The transport of neurotrophic factors from the brain to the cell body of neurons is essential to the survival of most ocular nerves. Deprivation of neurotrophic factors can induce apoptosis of neurons (Raff et al. 1993).

The neurotrophin (NT) family of peptides include nerve growth factor (NGF), brain-derived neurotrophic factor (BDNF), NT-3, NT-4/5 and NT-6. They act by binding to the neurotrophin receptors (NT-receptors), such as TrkA, TrkB, TrkC and p75NTR. The Trk receptors are tyrosine kinases. TrkA is selective for NGF, TrkB is selective for both BDNF and NT-4/5, whereas TrkC is selective for NT-3. After binding, the NT-receptor complex is internalized and transported via the axon to the soma. These receptors undergo ligand-induced phosphorylation and dimerization, and activate a cascade of Ras protein-mediated signal transduction events that affect multiple vital functions of the neuron (Lewin et al. 1997; Segal et al. 1996; Ebadi et al. 1997; Kaplan et al. 1997). Thus, these receptors play a fundamental role in the regulation of survival and differentiation of developing neurons and contribute to the maintenance of neuronal machinery in life.

In the ocular tissue, for example, mRNA of both TrkA and TrkB has been observed in retinal ganglion cells (RGC), dopaminergic amacrine cells and the optic nerve. Their expression was shown to be highly regulated during neuronal development (Jelsma et al. 1993; Rickman et al. 1995; Ugolini et al. 1995; Cellerino et al. 1997). The TrkB receptor-selective ligands, BDNF and NT-4/5, have been shown to be efficacious for the protection of RGC. Numerous studies have shown that these NTs not only improve the survival and neurite outgrowth of RGC in culture, but also significantly reduce axotomy-induced in vivo damage of the optic nerve and RGC, as well as stimulate the growth of axonal branches from regenerating RGC (Anderson et al. 1974; Quigley et al. 1976; Mansour-Robaey et al. 1994; Meyer-Franke et al. 1995; and Cui et al. 1994). For example, a single intravitreal injection of 5 μg of BDNF prevented the death of the axotomized ocular nerves when administered during the first five days after injury (Mansour Robaey 1994; Gao et al. 1997).

Ciliary neurotrophic factor (CNTF) and Basic Fibroblast Growth Factor (bFGF) are other neurotrophic factors that support survival of neurons. They are structurally unrelated to neurotrophins. They have also been shown to prevent lesion-induced death of neurons and axons (Mey et al. 1993; Weibel et al. 1995).

In normal human and rat corneas, neurotrophic factors, such as NGF, were found to be present (Lambiase et al. 2000). Human and rat corneal epithelial cells produce, store and release NGF and also express the TrkA receptor (Lambiase et al. 1998, Lambiase et al. 2000). These trophic factors appear to play an important role in the biology of the cornea. In the cornea of TrkA knockout mice, there was a drastic reduction in the number of nerve trunks, branches and thin nerve terminals. The blinking response of these mice to mechanical, thermal and chemical noxious stimuli was also significantly reduced (De Castro et al. 1998).

Thus, neurotrophic factors are important for the health and normal function of the cornea. These trophic factors, however, are peptide molecules, and are therefore difficult to exploit pharmaceutically due to bioavailability problems generally resident in the pharmaceutical administration of peptides. What are needed, therefore, are non-peptide molecules which stimulate neurotrophic activity in compromised retinal tissues, without the bioavailability problems attendant to the natural peptides.

### SUMMARY OF THE INVENTION

The present invention overcomes these and other drawbacks of the prior art by providing compositions and methods for treating conditions resulting from injury to corneal nerves. The compositions comprise one or more compound that promotes neuron regeneration or neurite outgrowth in a pharmaceutically acceptable vehicle.

As used herein, “compounds that promote neuron regeneration or neurite outgrowth” refers to those compounds which increase the in situ production or activity of neurotrophic factors in the ocular tissue, especially the cornea. As used herein, “neurotrophic factor” refers to NGF, BDNF, NT-3, NT-4/5, NT-6, CNTF, bFGF or other trophic factors which prevent, treat or ameliorate cornea neuropathy or promotes the re-growth of damaged cornea neurons. Examples of neurotrophic factor stimulators include: AIT-082 (neotrofin), idebenone, CB-1093, NS521 ((1-(1-butyl)-4-(2-oxo-1-benzimidazolone) piperidine), SS-701, and KT-711 (all shown below), ONO-2506, and clenbuterol. The most preferred neurotrophin stimulator of the present invention is AIT-082 (neotrofin). The preceding molecules may be obtained commercially or may be synthesized by methods known to those skilled in the art.

The methods of the present invention comprise administering to a human patient one or more compounds that promote neuron regeneration or neurite outgrowth, such as neurotrophic factor stimulators, for the treatment of conditions resulting from corneal nerve damage due to surgery.

The methods of the present invention are particularly directed to the use of neuron regeneration or neurite outgrowth promoting compounds for the treatment of dry eye, and other conditions resulting from corneal nerve damage, such as a decrease in corneal sensitivity.

The neuron regeneration or neurite outgrowth promoting compounds of the present invention may be contained in various types of pharmaceutical compositions, in accordance with formulation techniques known to those skilled in the art. In general, the neuron regeneration or neurite outgrowth promoting compounds will be formulated in solutions or suspensions for topical ophthalmic or intraocular administration, or as tablets, capsules or solutions for systemic administration (e.g., oral or intravenous). Preferably, the compounds of the invention will be formulated in a solution or suspension for topical ophthalmic application.

### DETAILED DESCRIPTION PREFERRED EMBODIMENTS

LASIK, and other vision-correction surgeries have allowed numerous corrective lens-wearing people to cease their use of corrective lenses. This is advantageous for many reasons. For people in some professions, such as art, science and construction work, corrective lenses can be a nuisance because of the dirt, paints, and chemicals with which they must work. However, patients frequently experience a decrease in corneal sensitivity and mild to moderate dry eye after LASIK surgery. In most patients, this is an acute problem lasting for only a few days. However, in a significant number of patients, the problem may persist for several months or more (Yu 2000). This problem is most likely the result of injury to the corneal nerves during surgery (Wilson 2001; Ambrosio & Wilson 2001). The present inventors have discovered that treatment of the injured corneal nerves after surgery with compounds that promote neurite outgrowth or that stimulate the regeneration of the severed or injured nerves can shorten the duration of, or reduce the incidence of, dry eye. Such treatment can also attenuate the decrease in corneal sensitivity caused by LASIK or other surgeries in which corneal nerves are damaged.

The present invention is directed at the use of compounds that promote the regeneration of severed nerves and/or neurite outgrowth to treat dry eye and the reduction in corneal sensitivity induced by cornea surgery. The compounds that promote the regeneration of severed neuron or promote neurite outgrowth do so by stimulating the production of, or by increasing the activity of, neurotrophic factors. The compounds used in the present invention may also promote the regeneration of severed nerves and/or neurite outgrowth by direct action on the injured nerves.

Several neurotrophic factor stimulators have been reported in the scientific literature, for example, AIT-082 (Graul & Castaner 1997), idebenone (Nabeshima et al. 1994), ONO-2506 (Matsui et al. 1998), NS521 (Gronborg et al. 1998), CB-1093 (Aimone et al. 1998) and Clenbuterol (Culmsee et al. 1998). However, nowhere in the art has it been disclosed or suggested to use neurotrophic factor stimulators to treat dry eye or other iatrogenic injury following Lasik surgery or other surgeries.

Topical ocular formulations of the neuron regeneration or neurite outgrowth promoting compounds are preferred due to ease of administration. Topical ocular formulations may be in solutions or suspensions. In general, topical formulations will contain the active neurotrophin factor stimulator and inert excipients.

The compositions of the present invention may be administered intraocularly following damage to the corneal nerve, such as by LASIK or other surgeries. Compositions useful for intraocular administration will generally be intraocular injection compositions or surgical irrigating solutions. Intraocular injection compositions will generally be comprised of an aqueous solution, e.g., balanced salt irrigating solutions, discussed below.

When the neuron regeneration or neurite outgrowth promoting compounds are administered after surgical procedures, such as through retrobulbar or periocular injection and intraocular perfusion or injection, the use of balanced salt irrigating solutions as vehicles are most preferred. BSS® Sterile Irrigating Solution and BSS Plus® Sterile Intraocular Irrigating Solution (Alcon Laboratories, Inc., Fort Worth, Tex., USA) are examples of physiologically balanced intraocular irrigating solutions. The latter type of solution is described in U.S. Pat. No. 4,550,022, the entire contents of which are incorporated herein by reference. Retrobulbar and periocular injections are known to those skilled in the art and are described in numerous publications including, for example, Ophthalmic Surgery: Principles of Practice (1990). The preferred route of administration is ocular topical application. Thus, pharmaceutically effective amounts of the above compounds or their active analogs in solutions or suspensions will be formulated for topical ophthalmic administration by methods known to those skilled in the art.

In general, the doses utilized for the above described purposes will vary, but will be in an effective amount to prevent, reduce or ameliorate the dry eye or decrease in cornea sensitivity related to surgery. As used herein, “pharmaceutically effective amount” refers to that amount of a neurotrophin factor stimulator which prevents, reduces or ameliorates the dry eye or decrease in cornea sensitivity related to surgery or trauma. The neurotrophic factor stimulators will generally be contained in the topical formulations or pharmaceutically acceptable carrier contemplated herein in an amount of from about 0.001 to about 10.0% weight/volume (% w/v). Preferred concentrations will range from about 0.1 to about 5.0% w/v. Topical formulations will generally be delivered to the eye one to six times a day, at the discretion of a skilled clinician. Systemic administration compositions will generally contain about 1-1000 mg of a neurotrophic factor stimulator, and can be taken 1-4 times per day, at the discretion of a skilled clinician.

As used herein, the term “pharmaceutically acceptable carrier” refers to any formulation which is safe, and provides the appropriate delivery of an effective amount of at least one neurotrophic factor stimulator for the desired route of administration.

The compositions of the present invention may contain additional pharmaceutically active agents or may be dosed concurrently with other pharmaceutical compositions. In particular, when treating a mammal for the prevention, treatment or amelioration of conditions resulting from injury to corneal nerves during surgery, the compositions of the present invention may contain additional agents or may be dosed concurrently or sequentially with other agents or compositions. Examples of agents include: artificial tear, artificial moisterizing solutions or other appropriate agents known to those skilled in the art.

### EXAMPLES

The following example demonstrates the protective efficacy of a neurotrophic factor stimulator (propentofylline) against ocular tissue cell insult.

### Example 1

The Compounds can be administered systemically or locally to the eye (e.g., topically, intracamerally, or via an implant). The Compounds are preferrably incorporated into topical ophthalmic formulations for delivery to the eye. The Compounds may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving a Compound in a physiologically acceptable isotonic aqueous buffer. Further, the ophthalmic solution may include an ophthalmologically acceptable surfactant to assist in dissolving the Compound. Furthermore, the ophthalmic solution may contain an agent to increase viscosity, such as, hydroxymethylcellulose, hydroxyethylcellulose, hydroxypropylmethylcellulose, methyl-cellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the active ingredient in a hydrophilic base prepared from the combination of, for example, carbopol-940, or the like, according to the published formulations for analogous ophthalmic preparations; preservatives and tonicity agents can be incorporated.

The Compounds are preferably formulated as topical ophthalmic suspensions or solutions, with a pH of about 5 to 8. The Compounds will normally be contained in these formulations in an amount 0.001% to 5% by weight, but preferably in an amount of 0.05% to 2% by weight. Thus, for topical presentation 1 to 2 drops of these formulations would be delivered to the surface of the eye 1 to 4 times per day according to the routine discretion of a skilled clinician.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

References

The following references, to the extent that they provide exemplary procedural or other details supplementary to those set forth herein, are specifically incorporated herein by reference.

United States Patents

4,550,022

Books

Ophthalmic Surgery: Principles of Practice, Ed., G. L. Spaeth, W.B. Sanders Co., Philadelphia, Pa., U.S.A., pages 85-87 (1990).

Other Publications

Aimone et al., The 1α,25(OH)₂D₃ analog CB-1093 induces nerve growth factor in non-human primate brain, SOCIETY FOR NEUROSCI. ABSTRACTS, 24:292, (1998).

Ambrosio & Wilson, J. Refractive Surgery 17:350-380 (2001).

Anderson et al., Effect of intraocular pressure on rapid axoplasmic transport in monkey optic nerve, INVEST. OPHTHALMOL., 13:771-783 (1974).

Beck et al., Brain-derived neurotrophic factor protects against ischemic cell damage in the rat hippocampus, J. CEREB. BLOOD FLOW METAB., 14:689-692 (1994).

Cellerino et al., Brain-derived neurotrophic factor/neurotrophin-4 receptor TrkB is localized on ganglion cells and dopaminergics amacrine cells in the vertebrate retina, J. COMP. NEUROL., 386:149-160 (1997).

Cui et al., NT-4/5 reduces naturally occurring retinal ganglion cell death in neonatal rats, NEUROREPORT, 5:1882-1884 (1994).

Culmsee et al., NGF antisense oligonucleotide blocks protective effects of clenbuterol against glutamate-induced excitotoxicity in vitro and focal cerebral ischemia in vivo, SOCIETY FOR NEUROSCI. ABSTRACTS, 24:295 (1998).

De Castro et al., Corneal innervation and sensitivity to noxious stimuli in trkA knockout mice, EUR. J. NEUROSCI., 10:146-152 (1998).

Ebadi et al., Neurotrophins and their receptors in nerve injury and repair, NEUROCHEM INT., 30:347-374 (1997).

Gao et al., Elevated mRNA expression of brain-derived neurotrophic factor in retinal ganglion cell layer after optic nerve injury, INVEST. OPHTHALMOL. VIS. SCI., 38:1840-1847 (1997).

Graul & Castaner, AIT-082, DRUGS OF THE FUTURE, 22:945-947 (1997).

Gronborg et al., Neuroprotection by a novel compound, NS521, SOCIETY FOR NEUROSCI. ABSTRACTS, 24:1551 (1998).

Jelsma et al., Different forms of the neurotrophin receptor trkB mRNA predominate in rat retina and optic nerve, J. NEUROBIOL., 24:1207-1214 (1993).

Kaplan et al., Signal transduction by the neurotrophin receptors, CURR. OPIN. CELL BIOL., 9:213-221 (1997).

Kirsch et al., Evidence for multiple, local functions of ciliary neurotrophic factor (CNTF) in retinal development: expression of CNTF and its receptors and in vitro effects on target cells, J. NEUROCHEM., 68:979-990 (1997).

Lambiase et al., Expression of nerve growth factor receptors on the ocular surface in healthy subjects and during manifestation of inflammatory diseases, INVEST. OPHTHALMOL. VIS. SCI.pb , 39:1272-1275 (1998).

Lambiase et al., Nerve growth factor promotes corneal healing: structural, biochemical, and molecular analyses of rat and human corneas, INVEST. OPHTHALMOL. VIS. SCI., 41:1063-1069 (2000).

Lewin et al., Physiology of the neurotrophins, ANN. REV. NEUROSCI., 19:289-317 (1997).

Lindholm et al., Brain-derived neurotrophic factor is a survival factor for cultured rat cerebellar granule neurons and protects them against glutamate-induced neurotoxicity, EUR. J. NEUROSCI., 5:1455-1464 (1993).

Mansour-Robaey et al., Effects of ocular injury and administration of brain-derived neurotrophic factor on survival and regrowth of axotomized retinal ganglion cells, PROC. NATL. ACAD. SCI. USA, 91:1632-1636 (1994).

Matsui et al., Protective effects of ONO-2506 on neurological deficits and brain infarct volume following 1 week of permanent occlusion of middle cerebral artery in rats, SOCIETY FOR NEUROSCI. ABSTRACTS, 24:254 (1998).

Mey et al., Intravitreal injections of neurotrophic factors support the survival of axotomized retinal ganglion cells in adult rats in vivo, BRAIN RES., 602:304-317 (1993).

Meyer-Franke et al., Characterization of the signaling interactions that promote the survival and growth of developing retinal ganglion cells in culture, NEURON, 15:805-819 (1995).

Nabeshima et al., Oral administration of NGF synthesis stimulators recovers reduced brain NGF content in aged rats and cognitive dysfunction in basal-forebrain-lesioned rats, GERONTOLOGY, 40(supp. 2):46-56 (1994).

Quigley et al., The dynamics and location of axonal transport blockade by acute intraocular pressure elevation in primate optic nerve, INVEST. OPHTHALMOL., 15:606-616 (1976).

Raff et al., Programmed cell death and the control of cell survival: lessons from the nervous system, SCIENCE, 262:695-700 (1993).

Rickman et al., Expression of the protooncogene, trk, receptors in the developing rat retina, VIS. NEUROSCI., 12:215-222 (1995).

Segal et al., Intracellular signaling pathways activated by neurotrophic factors, ANN. REV. NEUROSCI., 19:463-489 (1996).

Ugolini et al., TrkA, TrkB and p75 mRNA expression is developmentally regulated in the rat retina, BRAIN RES, 704:121-124 (1995).

Unoki et al., Protection of the rat retina from ischemic injury by brain-derived neurotrophic factor, ciliary neurotrophic factor, and basic fibroblast growth factor, INVEST. OPHTHALMOL. VIS. SCI., 35:907-915 (1994).

Weibel et al., Brain-derived neurotrophic factor (BDNF) prevents lession-induced axonal die-back in young rat optic nerve, BRAIN RES., 679:249-254 (1995).

Wilson, OPHTHALMOLOGY 108:1082-1087 (2001).

Yu, SYMPOSIUM ON CATARACT, IOL AND REFRACTORY SURGERY, Abstract 263 (2000).

We claim:

1. A method for the treatment of dry eye resulting from injury to corneal nerves, said method comprising administering to a patient in need thereof a therapeutically effective amount of a composition comprising at least one compound that promotes neuron regeneration or neurite outgrowth.

2. The method of claim 1, wherein the injury to corneal nerves results from surgery.

3. The method of claim 2, wherein the surgery is LASIK surgery.

4. The method of claim 1, wherein the compound may be selected from the group consisting of propentofylline, AIT-082 (neotrofin), idebenone, ONO-2506, CB-1093, NS521 (1-(1-butyl)-4-(2-oxo-1-benzimidazone) pireridine, eliprodil, SR57746A (xaliproden hydrochloride) or pharmaceutically acceptable analogs thereof.

5. The method of claim 4, wherein the compound is AIT-082.

6. The method of claim 4, wherein the compound is eliprodil.

7. A method for the treatment of decrease in cornea sensitivity resulting from injury to corneal nerves, said method comprising administering to a patient in need thereof a therapeutically effective amount of at least one composition comprising a compound that promotes neuron regeneration or neurite outgrowth.

8. The method of claim 7, wherein the injury to corneal nerves results from surgery.

9. The method of claim 8, wherein the surgery is LASIK surgery.

10. The method of claim 7, wherein the compound may be selected from the group consisting of propentofylline, AIT-082 (neotrofin), idebenone, ONO-2506, CB-1093, NS521 (1-(1-butyl)-4-(2-oxo-1-benzimidazone) pireridine, eliprodil, SR57746A (xaliproden hydrochloride) or pharmaceutically acceptable analogs thereof.

11. The method of claim 10, wherein the compound is AIT-082.

12. The method of claim 10, wherein the compound is eliprodil.

13. A method for the treatment of injury to corneal nerves comprising administering to a patient in need thereof a therapeutically effective amount of a composition comprising a compound that promotes neuron regeneration or neurite outgrowth.

14. The method of claim 13, wherein the injury to corneal nerves results from surgery.

15. The method of claim 14, wherein the surgery is LASIK surgery.

16. The method of claim 13, wherein the compound may be selected from the group consisting of propentofylline, AIT-082 (neotrofin), idebenone, ONO-2506, CB-1093, NS521 (1-(1-butyl)-4-(2-oxo-1-benzimidazone) pireridine, eliprodil, SR57746A (xaliproden hydrochloride) or pharmaceutically acceptable analogs thereof.

17. The method of claim 16, wherein the compound is AIT-082.

18. The method of claim 16, wherein the compound is eliprodil.
